(19) United States
(12) Patent Application Publication
(10) Pub. No.: US 2010/0030001 A1
(43) Pub. Date: Feb. 4, 2010

(54) Process for catalytically producing ethylene directly from acetic acid in a single reaction zone

(76) Inventors: Laiyuan Chen, Houston, TX (US); Josefina T. Chapman, Houston, TX (US); James H. Zink, League City, TX (US); Barbara F. Kimmich, League City, TX (US); Victor J. Johnston, Houston, TX (US); Jan Cornelis van der Waal, Delft (NL); Virginie Zuzaniuk, Krommenie (NL)

Correspondence Address: FERRELLS, PLLC, P. O. BOX 312, CLIFTON, VA, 20124-1706, US

(21) Appl. No.: 12/221,137

(22) Filed: Jul. 31, 2008

(51) Int. Cl. C07C 1/207

(52) U.S. Cl. 585/638

(57) ABSTRACT

A process for the selective production of ethylene by vapor phase reaction of acetic acid over a hydrogenating catalyst composition to form ethylene in a single reaction zone is disclosed and claimed. In an embodiment of this invention reaction of acetic acid and hydrogen over either a copper supported on iron oxide, copper-aluminum catalyst, cobalt supported on H-ZSM-5, ruthenium-cobalt supported on silica or cobalt supported on carbon selectively produces ethylene in a vapor phase at a temperature in the range of about 250° C. to 350° C.

### FILED OF THE INVENTION

The present invention relates generally to a process for the production of ethylene from acetic acid. More specifically, the present invention relates to a process in which acetic acid is directly converted to ethylene in a single reaction zone which may include a combination of individual catalysts. Without intending to be bound by any theory, it is believed the catalysts are capable of concurrently hydrogenating acetic acid and converting intermediates to ethylene with high selectivity and yields.

### BACKGROUND

There is a long felt need for an economically viable process to convert acetic acid to ethylene. Ethylene is an important commodity feedstock for a variety of industrial products; for example, ethylene can then be converted to a variety of polymer and other monomer products. Fluctuating natural gas and crude oil prices contribute to fluctuations in the cost of conventionally produced, petroleum or natural gas-sourced ethylene, making the need for alternative sources of ethylene all the greater when oil prices rise.

It has been reported that ethylene can be produced from various ethyl esters in the gas phase in the temperature range of 150-300° C. over zeolite catalysts. The types of ethyl esters that can be employed include ethyl esters of formic acid, acetic acid and propionic acid. See, for example, U.S. Pat. No. 4,620,050 to Cognion et al., where selectivity is reported to be acceptable.

U.S. Pat. No. 4,270,015 to Knifton describes obtaining ethylene involving a two-step process in which a mixture of carbon monoxide and hydrogen (commonly known as synthesis gas) is reacted with a carboxylic acid containing 2 to 4 carbon atoms to form the corresponding ethyl ester of said carboxylic acid which is subsequently pyrolyzed in a quartz reactor at elevated temperatures in the range of about 200° to 600° C. to obtain ethylene. The ethylene thus produced contains other hydrocarbons, particularly, ethane as an impurity. It was also reported therein that the concentration of ethane can reach high values, near 5% by pyrolyzing pure ethyl propionate at 460° C. More importantly, the conversion of the esters and yield of ethylene are reported to be very low.

U.S. Pat. No. 4,399,305 to Schreck describes obtaining high purity ethylene from ethyl acetate employing a cracking catalyst composed of a perfluorosulfonic acid resin commercially sold under the trademark NAFION® by E.I. DuPont de Nemours & Co.

On the other hand, Malinowski et al. Bull. Soc. Chim. Belg. (1985), 94(2), 93-5, disclose that reaction of substrates such as acetic acid on low-valent titanium heterogenized on support materials such as silica (SiO₂) or titania (TiO₂) resulted in a mixture of products including diethyl ether, ethylene and methane where selectivity is poor.

WO 2003/040037 discloses that crystalline microporous metalloalumino-phosphates (ELAPO), particularly, SAPO-type zeolites, such as SAPO-5, SAPO-11, SAPO-20, SAPO-18 and SAPO-34, having Si/Al ratio of 0.03-017 are useful as adsorbent or as a catalyst for the production of olefins from an oxygenated feedstock containing methanol, ethanol, n-propanol, isopropanol, C4-C20 alcohols, methyl ethyl ether, di-methyl ether, di-ethyl ether, di-isopropyl ether, formaldehyde, dimethyl carbonate, dimethyl ketone and/or acetic acid. A similar disclosure utilizes a silicoaluminophosphate molecular sieves comprising at least one intergrown phase of molecular sieve. It is reported that in this process a feedstock containing an oxygenate contacts a catalyst comprising the molecular sieve in a reaction zone of a reactor at conditions effective to produce light olefins, particularly ethylene and propylene. See U.S. Pat. No. 6,812,372 to Janssen et al. It is mentioned that such oxygenated feedstocks include acetic acid, but the disclosure appears to be limited to either methanol or dimethyl ether. See, also, U.S. Pat. No. 6,509,290 to Vaughn et al., which further discloses conversion of oxygenated feedstocks to olefins.

Bimetallic ruthenium-tin/silica catalysts have been prepared by reaction of tetrabutyl tin with ruthenium dioxide supported on silica. It has been reported that these catalysts exhibit different selectivities based on their content of tin/ruthenium ratio (Sn/Ru). Specifically it has been reported that the selectivity for the hydrogenolysis of ethyl acetate is quite different, which depends upon the Sn/Ru ratio in the catalyst. For instance, with ruthenium alone on SiO₂, the reaction is not selective: methane, ethane, carbon monoxide, carbon dioxide as well as ethanol and acetic acid are produced. Whereas, with low tin content, it has been reported that the catalysts are fairly selective for the formation of acetic acid, while at higher Sn/Ru ratios, ethanol is the only detected product. See Loessard et al., Studies in Surface Science and Catalysis (1989), Volume Date 1988, 48 (Struct. React. Surf.), 591-600.

The catalytic reduction of acetic acid has also been studied. For instance, Hindermann et al., J. Chem. Res., Synopses (1980), (11), 373, have disclosed the catalytic reduction of acetic acid on iron and on alkali-promoted iron. In their study they found that the reduction of acetic acid on alkali-promoted iron, followed at least two different routes depending on the temperature. For example, they found that at 350° C., the Piria reaction was predominant and gave acetone and carbon dioxide, as well as they observed decomposition products methane and carbon dioxide. Whereas the decomposition products were reduced at lower temperatures. On the other hand, at 300° C. a normal reduction reaction was observed resulting in the formation of acetaldehyde and ethanol.

From the foregoing it is apparent that existing processes do not have the requisite selectivity to ethylene or existing art specifies starting materials other than acetic acid which are expensive and/or intended to produce products other than ethylene.

### SUMMARY OF THE INVENTION

It has now been unexpectedly found that ethylene can be made on an industrial scale directly from acetic acid with high selectivity and yield. More particularly, this invention provides a process for the selective formation of ethylene from acetic acid comprising: hydrogenating acetic acid over a suitable hydrogenating catalyst in the presence of hydrogen and converting intermediates to form ethylene in a single reaction zone. As examples of such catalysts the following catalyst metals may be used: Copper, cobalt, ruthenium, nickel, aluminum, chromium, zinc, and a mixture thereof.

### DETAILED DESCRIPTION OF THE INVENTION

The invention is described in detail below with reference to numerous embodiments for purposes of exemplification and illustration only. Modifications to particular embodiments within the spirit and scope of the present invention, set forth in the appended claims, will be readily apparent to those of skill in the art.

Unless more specifically defined below, terminology as used herein is given its ordinary meaning. % and like terms refer to mole percent unless otherwise indicated.

“Conversion” is expressed as a mole percentage based on acetic acid in the feed.

“Selectivity” is expressed as a mole percent based on converted acetic acid. For example, if the conversion is 50 mole % and 50 mole % of the converted acetic acid is converted to ethylene, we refer to the ethylene selectivity as 50%. Selectivity is calculated from gas chromatography (GC) data as follows:

$$Ethylene Selectivity , \\ \% = 100 * \frac{m mol Ethylene out ( GC )}{\frac{Total m mol C out ( GC )}{2} - m mol AcOH out ( GC )}$$

Without intending to be bound by theory, it is believed the conversion of acetic acid to ethylene in accordance with the invention proceeds in accordance with one or more of the following chemical equations:

### Step 1a: Hydrogenation of Acetic Acid to Ethylene.

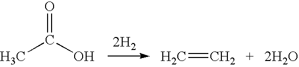

### Step 1b: Hydrogenation of Acetic Acid to Ethanol.

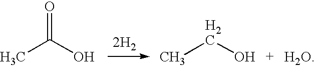

### Step 1c: Hydrogenation of Acetic Acid to Ethyl Acetate.

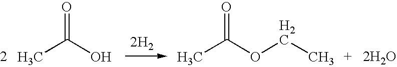

### Step 2a: Cracking of Ethyl Acetate to Ethylene and Acetic Acid.

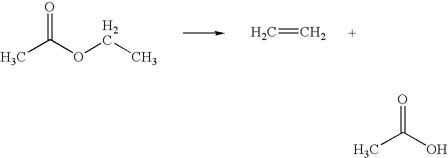

### Step 2b: Dehydration of Ethanol to Ethylene.

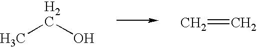

In accordance with the invention, conversion of acetic acid to ethylene is carried out in a single reaction zone which may be a single fixed bed, for example. The fixed bed can comprise a mixture of different catalyst particles or catalyst particles which include multiple catalysts. Typically, at least a hydrogenating catalyst is included in the reaction zone and optionally there is included a dehydrating and/or cracking catalyst as well.

Various hydrogenating catalysts known to one skilled in the art can be employed in hydrogenating acetic acid to ethanol in the first step of the process of this invention. The hydrogenating catalysts that are suitable are the ones which are metal catalysts on a suitable support. As noted earlier, examples of such catalysts the following catalysts may be mentioned without any limitation: Copper, cobalt, ruthenium, nickel, aluminum, chromium, zinc, palladium and a mixture thereof. Typically, a single metal, a bimetallic catalyst or a trimetallic catalyst on a suitable support can be used as a hydrogenating catalyst. Thus either copper alone or in combination with aluminum, chromium or zinc are particularly preferred. Similarly, cobalt alone or in combination with ruthenium is preferred. Examples of additional metals that can be used with cobalt as a second or third metal include without any limitation the following: platinum, palladium, rhodium, rhenium, iridium, chromium, copper, tin, molybdenum, tungsten and vanadium.

Various catalyst supports known in the art can be used to support the catalysts of this invention. Examples of such supports include without any limitation, zeolite, iron oxide, silica, alumina, titania, zirconia, magnesium oxide, calcium silicate, carbon, graphite and a mixture thereof. Preferred supports are H-ZSM-5, iron oxide, silica, calcium silicate, carbon or graphite. It is also important to note that higher the purity of silica better it is preferred as a support in this invention.

In an embodiment of this invention specific examples of supported hydrogenating catalysts include zeolite, such as H-ZSM-5, iron oxide, silica, alumina, titania, zirconia, magnesium oxide, calcium silicate, carbon, graphite and a mixture thereof. Particularly, as noted above, copper supported on iron oxide, copper-aluminum catalyst, cobalt supported on H-ZSM-5, a bimetallic catalyst ruthenium-cobalt supported on silica, cobalt supported on carbon are preferred.

A few of the commercially available catalysts include the following: copper-aluminum catalyst sold under the name of T-4489 by Sud Chemie; copper-zinc catalysts sold under the name of T-2130, T-4427 and T-4492; copper-chromium catalysts sold under the name of T-4419 and G-99B; and nickel catalysts sold under the name of NISAT 310, C47-7-04, G-49, and G-69; all sold by Sud Chemie. Copper-aluminum catalyst sold under the name of T-4489 is particularly preferred.

The amount of metal loading on a support is not very critical in this invention and can vary in the range of about 3 weight percent to about 10 weight percent. A metal loading of about 4 weight percent to about 6 weight percent based on the weight of the support is particularly preferred. Thus for example 4 to 6 weight percent of copper supported on iron oxide is particularly a preferred catalyst.

The metal impregnation can be carried out using any of the known methods in the art. Typically, before impregnation the supports are dried at 120° C. and shaped to particles having size distribution in the range of about 0.2 to 0.4 mm. Optionally the supports may be pressed, crushed and sieved to a desired size distribution. Any of the known methods to shape the support materials into desired size distribution can be employed.

For supports having low surface area, such as for example alpha-alumina or iron oxide, the metal solutions are added in excess until complete wetness or excess liquid impregnation so as to obtain desirable metal loadings.

As noted above, a few of the hydrogenating catalysts are bimetallic. Generally, in such cases, one metal acts as a promoter metal and the other metal is the main metal. For instance copper, nickel, cobalt and iron are considered to be main metals for preparing hydrogenating catalysts of this invention. The main metal can be combined with a promoter metal such as tungsten, vanadium, molybdenum, chromium or zinc. However, it should be noted that sometimes main metal can also act as a promoter metal or vice versa. For example, nickel can be used as a promoter metal when iron is used as a main metal. Similarly, chromium can be used as a main metal in conjunction with copper (i.e., Cu—Cr as main bimetallic metals), which can further be combined with promoter metals such as cerium, magnesium or zinc.

The bimetallic catalysts are generally impregnated in two steps. First, the “promoter” metal is added, followed by “main” metal. Each impregnation step is followed by drying and calcination. The bimetallic catalysts may also be prepared by co-impregnation. In the case of trimetallic Cu/Cr-containing catalysts as described above, a sequential impregnation may be used, starting with the addition of the “promoter” metal. The second impregnation step may involve co-impregnation of the two principal metals, i.e., Cu and Cr. For example, Cu—Cr—Ce on SiO₂ may be prepared by a first impregnation of Cerium nitrate, followed by the co-impregnation of copper and chromium nitrates. Again, each impregnation is followed by drying and calcinations. In most cases, the impregnation may be carried out using metal nitrate solutions. However, various other soluble salts which upon calcination releases metal ions can also be used. Examples of other suitable metal salts for impregnation include metal hydroxide, metal oxide, metal acetate, ammonium metal oxide, such as ammonium heptamolybdate hexahydrate, metal acids, such as perrhenic acid solution, metal oxalate, and the like.

As already noted above, in another aspect of the process of this invention, any of the known zeolites can be used as support catalysts. A wide variety of zeolite catalysts are known in the art including synthetic as well as natural, all of which can be used as support catalysts in this invention. More particularly, any zeolite having a pore diameter of at least about 0.6 nm can be used, preferably employed among such zeolites are the catalysts selected from the group consisting of mordenites, ZSM-5, a zeolite X and a zeolite Y.

The preparation of large-pore mordenites is described, for example, in U.S. Pat. No. 4,018,514 and in Mol. Sieves Pap. Conf., 1967, 78, Soc. Chem. Ind. London, by D. DOMINE and J. QUOBEX.

Zeolite X is described, for example, U.S. Pat. No. 2,882,244 and zeolite Y in U.S. Pat. No. 3,130,007.

Various zeolites and zeolite-type materials are known in the art for the catalysis of chemical reactions. For example, U.S. Pat. No. 3,702,886, of Argauer, discloses a class of synthetic zeolites, characterized as “Zeolite ZSM-5”, which are effective for the catalysis of various hydrocarbon conversion processes.

The zeolites suitable for the procedure of the invention can be in the basic form, in the partially or totally acidified form, or in the partially dealuminated form.

In another aspect of the process of this invention, any of known dehydration catalysts can be employed in the reaction zone of the process of this invention. Typically, a zeolite catalyst is employed as a dehydration catalyst and may support a dehydrogenating catalyst. While any zeolite having a pore diameter of at least about 0.6 nm can be used, preferably employed among such zeolites are the dehydration catalyst selected from the group consisting of mordenites, ZSM-5, a zeolite X and a zeolite Y.

An active dehydrating catalyst in the process of the present invention, characterized as “H-ZSM-5” or “H-mordenite” zeolites are prepared from a corresponding “ZSM-5” zeolite or “mordenite” zeolite by replacing most, and generally at least about 80% of the cations of the latter zeolite with hydrogen ions using techniques well-known in the art. H-Mordenite zeolite, for example, was prepared by calcination of ammonium form Mordenite at 500-550° C. for 4-8 hours. If the sodium form of Mordenite is used as a precursor, the sodium Mordenite is ion-exchanged to ammonium form prior to calcination.

These zeolite catalysts are essentially crystalline aluminosilicates or in the neutral form a combination of silica and alumina in a well defined crystalline structure. In a particularly preferred class of zeolite catalysts for purposes of the present invention, the molar ratio of SiO₂ to Al₂O₃ in these zeolites is within the ratio of about 10 to 60.

As noted earlier, ethylene is produced by dehydration as well as the decomposition or “cracking” of ethyl acetate to ethylene and acetic acid. This may simply occur as thermal cracking at elevated temperatures or may be a catalyzed reaction if so desired, utilizing a cracking catalyst. Suitable cracking catalysts include sulfonic acid resins such as perfluorosulfonic acid resins disclosed in U.S. Pat. No. 4,399,305 noted above, the disclosure of which is incorporated by reference. Zeolites are also suitable as cracking catalysts as noted in U.S. Pat. No. 4,620,050, the disclosure of which is also incorporated by reference. Thus, a zeolite catalyst may be used to concurrently dehydrate ethanol to ethylene and decompose ethyl acetate to ethylene in a highly efficient process of the invention.

Selectivities of acetic acid to ethylene are suitably more than 10% an more such as at least 20%, at or least 25% or so up to about 40% in typical cases Depending on the by-product mix, it may be desirable to operate at intermediate selectivities, and recirculate products such as acetaldehyde for further hydrogenating and dehdration provided selectivity to undesirable products such as CO₂ remains low.

Preferably, for the purposes of the process of this invention, the suitable hydrogenating catalyst is either copper on iron oxide or copper-aluminum catalyst, sold under the tradename of T-4489 by Sud Chemie, cobalt supported on H-ZSM-5, a bimetallic catalyst, ruthenium and cobalt supported on silica, and cobalt supported on carbon. In this embodiment of the process of this invention, the copper loading on the iron oxide support or in the bimetallic copper-aluminum catalyst is typically in the range of about 3 weight percent to about 10 weight percent, preferably it is in the range of about 4 weight percent to about 6 weight percent. Similarly, the loading of cobalt on H-ZSM-5 or silica or carbon is typically around 5 weight percent. The amount of ruthenium in the bimetallic catalyst is also around 5 weight percent.

In another aspect of the process of this invention, the acetic acid hydrogenation and dehydration are carried out at a pressure just sufficient to overcome the pressure drop across the catalytic bed.

The reaction may be carried out in the vapor or liquid state under a wide variety of conditions. Preferably, the reaction is carried out in the vapor phase. Reaction temperatures may be employed, for example in the range of about 200° C. to about 375° C., preferably about 250° C. to about 350° C. The pressure is generally uncritical to the reaction and subatmospheric, atmospheric or superatmospheric pressures may be employed. In most cases, however, the pressure of the reaction will be in the range of about 1 to 30 atmospheres absolute.

Although the reaction consumes two moles of hydrogen per mole of acetic acid to produce a mole of ethylene, the actual molar ratio of acetic acid to hydrogen in the feed stream may be varied between wide limits, e.g. from about 100:1 to 1:100. It is preferred however that such ratio be in the range of about 1:20 to 1:2.

The raw materials used in connection with the process of this invention may be derived from any suitable source including natural gas, petroleum, coal, biomass and so forth. It is well known to produce acetic acid through methanol carbonylation, acetaldehyde oxidation, ethylene oxidation, oxidative fermentation, and anaerobic fermentation and so forth. As petroleum and natural gas have become more expensive, methods for producing acetic acid and intermediates such as methanol and carbon monoxide from alternate carbon sources have drawn more interest. Of particular interest is the production of acetic acid from synthesis gas (syngas) that may be derived from any suitable carbon source. U.S. Pat. No. 6,232,352 to Vidalin, the disclosure of which is incorporated herein by reference, for example, teaches a method of retrofitting a methanol plant for the manufacture of acetic acid. By retrofitting a methanol plant the large capital costs associated with CO generation for a new acetic acid plant are significantly reduced or largely eliminated. All or part of the syngas is diverted from the methanol synthesis loop and supplied to a separator unit to recover CO and hydrogen, which are then used to produce acetic acid. In addition to acetic acid, the process can also be used to make hydrogen which is utilized in connection with this invention.

U.S. Pat. No. RE 35,377 Steinberg et al., also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syngas is converted to methanol which may be carbonylated to acetic acid. The method likewise produces hydrogen which may be used in connection with this invention as noted above. See also, U.S. Pat. No. 5,821,111 Grady et al., which discloses a process for converting waste biomass through gasification into synthesis gas as well as U.S. Pat. No. 6,685,754 Kindig et al., the disclosures of which are incorporated herein by reference.

The acetic acid may be vaporized at the reaction temperature, and then it can be fed along with hydrogen in undiluted state or diluted with a relatively inert carrier gas, such as nitrogen, argon, helium, carbon dioxide and the like.

Alternatively, acetic acid in vapor form may be taken directly as crude product from the flash vessel of a methanol carbonylation unit of the class described in U.S. Pat. No. 6,657,078 of Scates et al., the disclosure of which is incorporated by reference. The crude vapor product may be fed directly to the reaction zones of the present invention without the need for condensing the acetic acid and light ends or removing water, saving overall processing costs.

Contact or residence time can also vary widely, depending upon such variables as amount of acetic acid, catalyst, reactor, temperature and pressure. Typical contact times range from a fraction of a second to more than several hours when a catalyst system other than a fixed bed is used, with preferred contact times, at least for vapor phase reactions, between about 0.5 and 100 seconds.

Typically, the catalyst is employed in a fixed bed reactor e.g. in the shape of an elongated pipe or tube where the reactants, typically in the vapor form, are passed over or through the catalyst. Other reactors, such as fluid or ebullient bed reactors, can be employed, if desired. In some instances, it is advantageous to use the catalyst bed in conjunction with an inert material such as glass wool to regulate the pressure drop of the reactant stream through the catalyst bed and the contact time of the reactant compounds with the catalyst particles.

In one of the preferred embodiments there is also provided a process for selective formation of ethylene from acetic acid comprising: contacting a feed stream of acetic acid and hydrogen at a temperature in the range of about 250° C. to 350° C. with a catalyst chosen from copper supported on iron oxide, copper-aluminum catalyst, cobalt supported on H-ZSM-5, ruthenium-cobalt supported on silica or cobalt supported on carbon to form ethylene.

In this embodiment of the process of this invention, the preferred catalyst is 5 weight percent copper on iron oxide, 5 weight percent cobalt on H-ZSM-5, 5 weight percent cobalt and 5 weight percent ruthenium on silica or 5 weight percent cobalt on carbon. In this embodiment of the process of this invention it is preferred that the reaction is carried out in the vapor phase in a tubular reactor packed with the catalyst bed and at a temperature in the range of about 250° C. to 350° C. and at a pressure in the range of about 1 to 30 atmospheres absolute, and the contact time of reactants is in the range of about 0.5 and 100 seconds.

The following examples describe the procedures used for the preparation of various catalysts employed in the process of this invention.

### EXAMPLE A

### Preparation of 5 Weight Percent Copper on Iron Oxide

Powdered and meshed iron oxide (100 g) of uniform particle size distribution of about 0.2 mm was dried at 120° C. in an oven under nitrogen atmosphere overnight and then cooled to room temperature. To this was added a solution of copper nitrate pentahydrate (17 g) in distilled water (100 ml). The resulting slurry was dried in an oven gradually heated to 110° C. (>2 hours, 10° C./min.). The impregnated catalyst mixture was then calcined at 500° C. (6 hours, 1° C./min).

### EXAMPLE B

### Preparation of 5 Weight Percent Cobalt on H-ZSM-5

Example A is substantially repeated with the exception of using appropriate amount of cobalt nitrate hexahydrate as the metal salt and H-ZSM-5 as the support catalyst to prepare 5 weight percent cobalt supported on H-ZSM-5.

### EXAMPLE C

### Preparation of 5 Weight Percent Cobalt and 5 Weight Percent Ruthenium on Silica

Example A is substantially repeated with the exception of using lo appropriate amounts of cobalt nitrate hexahydrate and ruthenium nitrosyl nitrate as the metal salts and silica as the support catalyst to prepare 5 weight percent cobalt and 5 weight percent ruthenium supported on silica.

### EXAMPLE D

### Preparation of 5 Weight Percent Cobalt on Carbon

Example A is substantially repeated with the exception of using appropriate amount of cobalt nitrate hexahydrate as the metal salt and carbon as the support catalyst to prepare 5 weight percent cobalt supported on carbon.

### Gas Chromatographic (GC) Analysis of the Products

The analysis of the products was carried out by online GC. A three channel compact GC equipped with one flame ionization detector (FID) and 2 thermal conducting detectors (TCDs) was used to analyze the reactants and products. The front channel was equipped with an FID and a CP-Sil 5 (20 m)+WaxFFap (5 m) column and was used to quantify:

Acetaldehyde

Ethanol

Acetone

Methyl acetate

Vinyl acetate

Ethyl acetate

Acetic acid

Ethylene glycol diacetate

Ethylene glycol

Ethylidene diacetate

Paraldehyde

The middle channel was equipped with a TCD and Porabond Q column and was used to quantify:

CO₂

Ethylene

Ethane

The back channel was equipped with a TCD and Molsieve 5A column and was used to quantify:

Helium

Hydrogen

Nitrogen

Methane

Carbon monoxide

Prior to reactions, the retention time of the different components was determined by spiking with individual compounds and the GCs were calibrated either with a calibration gas of known composition or with liquid solutions of known compositions. This allowed the determination of the response factors for the various components.

### EXAMPLE 1

The catalyst utilized was 5 weight percent copper on iron oxide prepared in accordance with the procedure of Example A

In a tubular reactor made of stainless steel, having an internal diameter of 30 mm and capable of being raised to a controlled temperature, there are arranged 50 ml of 5 weight percent copper on iron oxide catalyst. The length of the catalyst bed after charging was approximately about 70 mm.

A feed liquid was comprised essentially of acetic acid. The reaction feed liquid was evaporated and charged to the reactor along with hydrogen and helium as a carrier gas with an average combined gas hourly space velocity (GHSV) of about 2500 hr⁻¹ at a temperature of about 350° C. and pressure of 100 psig. The resulting feed stream contained a mole percent of acetic acid from about 4.4% to about 13.8% and the mole percent of hydrogen from about 14% to about 77%. A portion of the vapor effluent was passed through a gas chromatograph for analysis of the contents of the effluents. Results appear in Table 1. The selectivity to ethylene was 16% at an acetic acid conversion of 100%

### EXAMPLE 2

The catalyst utilized was 5 weight percent cobalt on H-ZSM-5 prepared in accordance with the procedure of Example B.

The procedure as set forth in Example 1 was substantially repeated with an average combined gas hourly space velocity (GHSV) of 10,000 hr⁻¹ of the feed stream of the vaporized acetic acid, hydrogen and helium at a temperature of 250° C. and pressure of 1 bar. A portion of the vapor effluent was passed through a gas chromatograph for analysis of the contents of the effluents. Results appear in Table 1. The acetic acid conversion was 3% and ethylene selectivity was 28%.

### EXAMPLE 3

The catalyst utilized was a bimetallic catalyst containing 5 weight percent cobalt and 5 weight percent ruthenium supported on silica prepared in accordance with the procedure of Example C.

The procedure as set forth in Example 1 was substantially repeated with an average combined gas hourly space velocity (GHSV) of 2500 hr⁻¹ of the feed stream of the vaporized acetic acid, hydrogen and helium at a temperature of 350° C. and pressure of 1 bar. A portion of the vapor effluent was passed through a gas chromatograph for analysis of the contents of the effluents. Results appear in Table 1. The acetic acid conversion was 4% and ethylene selectivity was 14%.

### EXAMPLE 4

The catalyst utilized was 5 weight percent cobalt supported on carbon prepared in accordance with the procedure of Example D.

The procedure as set forth in Example 1 was substantially repeated with an average combined gas hourly space velocity (GHSV) of 2500 hr⁻¹ of the feed stream of the vaporized acetic acid, hydrogen and helium at a temperature of 350° C. and pressure of 1 bar. A portion of the vapor effluent was passed through a gas chromatograph for analysis of the contents of the effluents. Results appear in Table 1. The acetic acid conversion was 2% and ethylene selectivity was 12%.

Generally speaking, selectivities to ethylene above 10% or so are highly desirable; it being appreciated that the other by-products such as ethanol or ethyl acetate can be re-cycled to the reactor along with unreacted acetic acid, while still other by-products can be re-processed or used for fuel value. Selectivities to CO₂ of less than 10% are desired, preferably 5% or less.

TABLE 1
Acetic Acid Conversion and Selectivities

| | Ethylene | Acetic acid | |
|---|---|---|---|
| | selectivity | conversion | |
| Example | (%) | (%) | Other products |
| 1 | 16 | 100 | acetaldehyde-31%, ethane-15%, |
| | | | ethyl acetate-4%, CO₂-5% |
| 2 | 29 | 3 | acetaldehyde-51%, ethane-28% |
| 3 | 14 | 4 | acetaldehyde-78%, ethane-8% |
| 4 | 12 | 2 | acetone-8%, methane-47%, |
| | | | ethane-5% |

### COMPARATIVE EXAMPLES 1-5

These examples illustrate the reaction of acetic acid and hydrogen over a variety of catalysts wherein either no ethylene was formed and/or very low levels of ethylene was detected.

In all of these examples the procedure as set forth in Example 1 was substantially followed with the exception of using different catalysts as listed in Table 2. The reaction temperature and selectivity to ethylene are also tabulated in Table 2.

TABLE 2

| | | | Mol % | |
|---|---|---|---|---|
| | Reactor | | Acetic | |
| | Temperature | Mol % | Acid In | Ethylene |
| Catalyst | (° C.) | H₂ In Feed | Feed | Selectivity |
| 0.5%-1% Pd | 250-350° C. | 54.2% | 7.3% | 0% |
| on Carbon | | | | |
| 1% Ru on | 250-350° C. | 36.8% | 7.3% | 0% |
| Carbon | | | | |
| 2% Pt on | 350° C. | 34.3%-76.5% | 4.4%-7.3% | 0%-1% |
| Fe₂O₃ | | | | |
| 2.58% Pd/ | 250-350° C. | 36.8% | 7.3% | 0%-0.5% |
| 5.05% Mo on | | | | |
| SiO₂ | | | | |
| 4.79% Cu | 400° C. | 35.2% | 7.5% | 0%-2.25% |
| on SiO2 | | | | |

In these examples various other products including acetaldehyde, ethanol, ethyl acetate, ethane, carbon monoxide, carbon dioxide, methane, isopropanol, acetone and water were detected.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A process for selective and direct formation of ethylene from acetic acid comprising: contacting a feed stream containing acetic acid and hydrogen at an elevated temperature with a suitable hydrogenating catalyst in a single reaction zone to form ethylene, the reaction zone optionally including a dehydrating catalyst or a cracking catalyst.

2. The process according to claim 1, wherein the hydrogenation is carried out over a hydrogenating catalyst on a support, which catalyst is selected from the group consisting of copper, cobalt, ruthenium, nickel, aluminum, chromium, zinc, palladium and a mixture thereof.

3. The process according to claim 2, wherein the support is selected from the group consisting of iron oxide, H-ZSM-5, silica, alumina, titania, zirconia, magnesium oxide, calcium silicate, carbon, graphite and a mixture thereof.

4. The process according to claim 2, wherein the hydrogenating catalyst is selected from the group consisting of copper supported on iron oxide, copper-aluminum catalyst, copper-zinc catalyst, copper-chromium catalyst, cobalt supported on H-ZSM-5, ruthenium-cobalt supported on silica, cobalt supported on carbon and nickel catalyst.

5. The process according to claim 2, wherein the hydrogenating catalyst is chosen from copper supported on iron oxide, copper-aluminum catalyst, cobalt supported on H-ZSM-5, ruthenium-cobalt supported on silica or cobalt supported on carbon.

6. The process according to claim 1, wherein the hydrogenating catalyst is copper supported on iron oxide, cobalt supported on H-ZSM-5, ruthenium-cobalt supported on silica or cobalt supported on carbon.

7. The process according to claim 6, wherein the catalyst is copper supported on iron oxide.

8. The process according to claim 6, wherein the catalyst is cobalt supported on H-ZSM-5.

9. The process according to claim 6, wherein the catalyst is ruthenium-cobalt supported on silica or cobalt supported on carbon.

10. The process according to claim 6, wherein the loading of copper on iron oxide is in the range of about 3 weight percent to about 10 weight percent.

11. The process according to claim 7, wherein the loading of copper on iron oxide is in the range of about 4 weight percent to about 6 weight percent.

12. The process according to claim 6, wherein the loading of cobalt on H-ZSM-5, silica or carbon is in the range of about 3 weight percent to about 10 weight percent.

13. The process according to claim 12, wherein the loading of cobalt on H-ZSM-5, silica or carbon is in the range of about 4 weight percent to about 6 weight percent.

14. The process according to claim 6, wherein the loading of ruthenium on silica is in the range of about 3 weight percent to about 10 weight percent.

15. The process according to claim 6, wherein the loading of ruthenium on silica is in the range of about 4 weight percent to about 6 weight percent.

16. The process according to claim 1, wherein the hydrogenation is carried out at a pressure just sufficient to overcome the pressure drop across the catalytic bed.

17. The process according to claim 1, wherein the hydrogenation is carried out in the vapor phase and at a temperature in the range of about 200° to 375° C.

18. The process according to claim 17, wherein the hydrogenation is carried out in the vapor phase and at a temperature in the range of about 250° to 350° C.

19. The process according to claim 17, wherein the catalyst is in the form of a fixed layered bed and said feed stream into said bed also contains an inert carrier gas.

20. The process according to claim 17, wherein the reactants consists of acetic acid and hydrogen with a molar ratio in the range of about 100:1 to 1:100, the temperature of reaction is in the range of about 250° C. to 350° C., the pressure of reaction is in the range of about 1 to 30 atmospheres absolute and the contact time of reactants and catalyst is in the range of about 0.5 to 100 seconds.

21. The process according to claim 17, wherein the reactants consists of acetic acid and hydrogen with a molar ratio in the range of about 1:20 to 1:2, the temperature of reaction is in the range of about 300° C. to 350° C., the pressure of reaction is in the range of about 1 to 30 atmospheres absolute and the contact time of reactants and catalyst is in the range of about 0.5 to 100 seconds.

22. A process for selective formation of ethylene from acetic acid comprising: contacting a feed stream of acetic acid and hydrogen at a temperature in the range of about 250° C. to 350° C. with a hydrogenating catalyst chosen from copper supported on iron oxide, copper-aluminum catalyst, cobalt supported on H-ZSM-5, ruthenium-cobalt supported on silica or cobalt supported on carbon to form ethylene.

23. The process according to claim 22, wherein the hydrogenating catalyst is 5 weight percent copper on iron oxide.

24. The process according to claim 22, wherein the hydrogenating catalyst is 5 weight percent cobalt supported on H-ZSM-5.

25. The process according to claim 22, wherein the hydrogenation and dehydration catalysts are layered in a fixed bed and the reaction is carried out in the vapor phase and at a temperature in the range of about 300° C. to 350° C. and at a pressure in the range of about 1 to 30 atmospheres absolute, and the contact time of reactants is in the range of about 0.5 and 100 seconds.
